(19) United States
(12) Patent Application Publication
(10) Pub. No.: US 2014/0067062 A1
(43) Pub. Date: Mar. 6, 2014

(54) SOFT TISSUE GRAFT AND METHOD

(71) Applicant: M. Mary Sinnott, Logan, UT (US)

(72) Inventor: M. Mary Sinnott, Logan, UT (US)

(73) Assignee: MTP Solutions, LLC, North Logan, UT (US)

(21) Appl. No.: 13/602,577

(22) Filed: Sep. 4, 2012

(51) Int. Cl. A61F 2/08

(52) U.S. Cl. 623/13.15

(57) ABSTRACT

Soft tissue grafts and methods for making and using them are presented. The graft includes an intermediate portion and a reduced area end portion.

### FIELD OF THE INVENTION

The invention relates soft tissue grafts and methods for making and using them.

### BACKGROUND

Various conditions may affect skeletal joints such as the elongation, shortening, or rupture of soft tissues associated with the joint. Joint function may be restored by reconstruction of the soft tissues associated with the joint.

### SUMMARY

The present invention provides soft tissue grafts and methods for making and using them.

In one aspect of the invention, a soft tissue graft includes an intermediate portion and an end portion. The intermediate portion has a cross-sectional area. The end portion has a cross-sectional area less than the intermediate portion cross-sectional area. The end portion may have a cross-sectional area less than half that of the intermediate portion such that the end portion may be doubled over a passer and passed through a bone tunnel that fits closely around the intermediate portion. The graft may include a flexible tubular jacket and a flexible core disposed within the jacket. The jacket may be continuous between the intermediate portion and the end portion while the core may occupy the intermediate portion and be absent, or reduced in cross-sectional area, at the end portion such that the jacket is allowed to collapse to a smaller cross-sectional area in the end portion than in the intermediate portion. The intermediate portion and end portion may contain one or more branches. The graft may have one or more end portions. The jacket may be porous or non-porous. The jacket may be a film, expanded matrix, or a textile. For example, a film may be a thin continuous material; an expanded matrix may be a material that has been processed to introduce pores or to separate into interconnected fibrils such as by stretching and heating or crosslinking to form interconnected fibrils; and a textile may include fibers formed into a flexible network such as by weaving, braiding, knitting, bonding, or other textile process. The core may likewise be a film, expanded matrix, or textile as described above as well as twisted or parallel fibers. The jacket and core may be made of natural or synthetic biocompatible materials such as biological tissue, polymers, and other suitable materials and combinations thereof. For example, the core may include a structure to resist axial stretching and the jacket may include a structure that encourages tissue ingrowth. For example, the core may include longitudinal tows of unbraided fibers and the jacket may include braided fibers. In another example, the core may have fibers that resist axial stretching combined with bioactive bone growth inducing material. In another example, the bone inducing material may be positioned near the ends of the intermediate portion but not in the middle to encourage attachment of the graft via bone growth near the ends of the intermediate portion but not in the middle.

In another aspect of the invention, a method of making a soft tissue graft having an end portion with a cross-sectional area less than an intermediate portion cross-sectional area includes forming a graft with a flexible tubular jacket and a flexible core disposed within the jacket. The core is reduced, or removed, at the end portion and the jacket is collapsed to a smaller cross-sectional area in the end portion than in the intermediate portion.

In another aspect of the invention, a method of using a soft tissue graft having an end portion with a cross-sectional area less than an intermediate portion cross-sectional area includes inserting the end portion into a bone tunnel and advancing the end portion to position the intermediate portion adjacent to, or part-way into, the bone tunnel. The end portion may have a cross-sectional area less than half that of the intermediate portion and a method may include folding the end portion over a graft passer and passing the folded end portion through a bone tunnel.

### BRIEF DESCRIPTION OF THE DRAWINGS

Various examples of the present invention will be discussed with reference to the appended drawings. These drawings depict only illustrative examples of the invention and are not to be considered limiting of its scope.

FIG. 1 is a side elevation view of the human foot illustrating anatomic reference planes;

FIG. 2 is a dorsal view of the metatarsus and phalanx of the right second metatarsophalangeal joint of the human foot;

FIG. 3 is a medial view of the bones of FIG. 2;

FIG. 4 is a lateral view of the bones of FIG. 2;

FIG. 5 is a top plan view of an illustrative example of a soft tissue graft according to the present invention;

FIG. 6 is a cross-sectional view taken along line 6-6 of FIG. 5;

FIG. 7 is a cross-sectional view taken along line 7-7 of FIG. 5;

FIG. 8 is a top plan view of an illustrative example of a precursor material used to form the soft tissue graft of FIG. 2;

FIG. 9 is a top plan view of an illustrative step in the formation of the soft tissue graft of FIG. 2;

FIG. 10 is a top plan view of an illustrative step in the formation of the soft tissue graft of FIG. 2;

FIG. 11 is a top plan view of an illustrative step in the formation of the soft tissue graft of FIG. 2;

FIG. 12 is a top plan view of an illustrative step in the formation of the soft tissue graft of FIG. 2;

FIG. 13 is a top plan view of an illustrative step in the formation of the soft tissue graft of FIG. 2;

FIG. 14 is a side elevation view of an illustrative step in a soft tissue reconstruction procedure utilizing the soft tissue graft of FIG. 2;

FIG. 15 is a side elevation view of an illustrative step in a soft tissue reconstruction procedure utilizing the soft tissue graft of FIG. 2;

FIG. 16 is a side elevation view of an illustrative step in a soft tissue reconstruction procedure utilizing the soft tissue graft of FIG. 2; and

FIG. 17 is a side elevation view of an illustrative step in a soft tissue reconstruction procedure utilizing the soft tissue graft of FIG. 2.

### DESCRIPTION OF THE ILLUSTRATIVE EXAMPLES

The following illustrative examples illustrate soft tissue grafts and methods for making and using them. Grafts and methods according to the present invention may be used in conjunction with any graft procedure but the illustrative examples are shown in a size and form most suitable for reconstructing the soft tissues of joints of the hand and foot. In particular, the illustrative examples depict their use on metatarsophalangeal (MTP) joints of the human foot. The illustrative grafts and methods are also suitable for use on metacarpophalangeal (MCP) joints of the human hand.

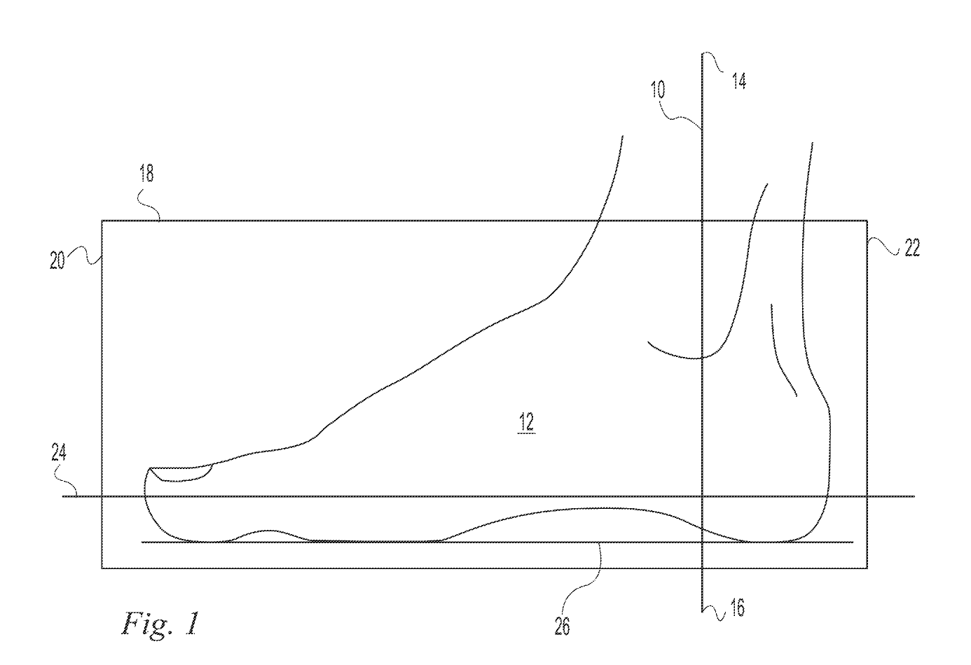

FIG. 1 illustrates the anatomic planes of the foot that are used for reference in this application. The coronal plane 10 extends from the medial aspect 12 to the lateral aspect of the foot and from dorsal 14 to plantar 16 and divides the foot between the toes and heel. The sagittal plane 18 extends anterior 20 to posterior 22 and dorsal 14 to plantar 16 and divides the foot into medial and lateral halves. The transverse plane 24 extends anterior 20 to posterior 22 and medial to lateral parallel to the floor 26.

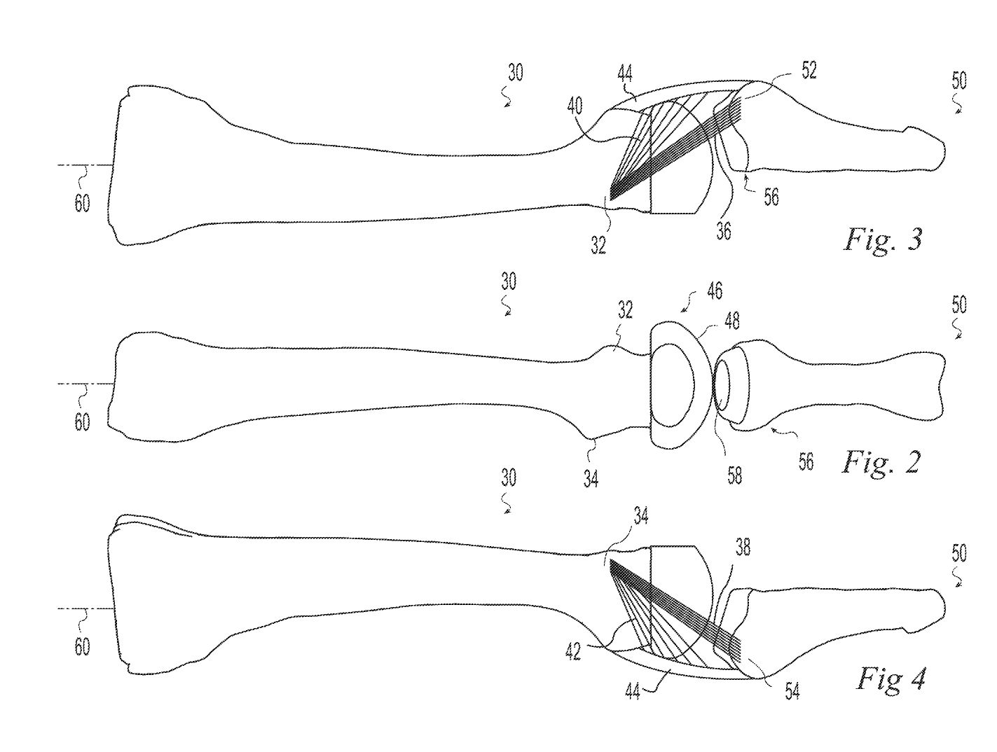

FIGS. 2-4 illustrate the metatarsus 30 and proximal phalanx 50 of the second MTP joint of the right foot. The medial and lateral epicondyles 32, 34, located on the medial-dorsal and lateral-dorsal aspects of the metatarsus 30 respectively, are the origins of the medial and lateral proper collateral ligaments (PCLs) 36, 38 and the medial and lateral accessory collateral ligaments (ACLs) 40, 42 of the MTP joint. The medial PCL inserts at the medial-plantar aspect 52 and the lateral PCL inserts at the lateral-plantar aspect 54 of the proximal phalanx 50. The ACLs fan out and insert into the plantar plate 44. The metatarsus includes a metatarsal head 46 having an articular surface 48 and the proximal phalanx includes a phalangeal head 56 having an articular surface 58. The metatarsus 30 further includes a longitudinal axis 60 extending lengthwise down the center of the bone.

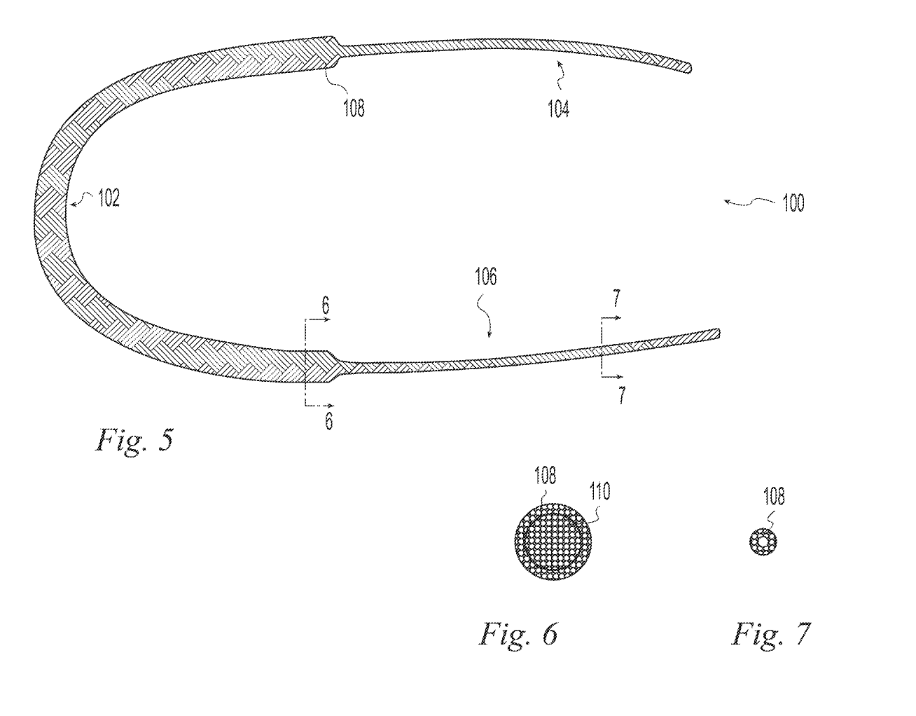

FIGS. 5-7 illustrate an exemplary soft tissue graft 100 according to the present invention. The graft 100 includes an intermediate portion 102 and end portions 104, 106 extending from each end of the intermediate portion 102. The intermediate portion 102 has an intermediate portion cross-sectional area and the end portions 104, 106 have end portion cross-sectional areas less than the intermediate portion cross-sectional area. The graft includes a jacket 108 extending along its length and a core 110 spanning the intermediate portion 102. In the illustrative example of FIGS. 5-7, the jacket 108 is formed from biocompatible fibers braided into a tubular, flexible sleeve and the core 110 is formed from parallel biocompatible fibers. In the illustrative example of FIGS. 5-7, the jacket 108 is continuous and has the same uniform construction in the intermediate and end portions. The core 110 is absent from the end portions thereby allowing the jacket to assume a smaller cross-sectional area in the end portions. In the illustrative example of FIGS. 5-7, the jacket 108 and core 110 are separate. Preferably, the end portion cross-sectional areas are less than or equal to one-half of the intermediate portion cross-sectional area such that the end portion can be doubled over and not exceed the cross-sectional area of the intermediate portion. The absence of the core 110 in the end portions results in the end portions being more flexible than the intermediate portion and further enhances the ability of the end portions to be doubled over and passed, e.g., through a bone tunnel. In the illustrative example of FIGS. 5-7, the graft has been shown with two end portions, one extending from each end of the intermediate portion. It is within the scope of the invention to form a graft with a single reduced area end portion extending from only one end of the intermediate portion where it is only necessary or desirable to pass the graft via one end. It is also within the scope of the invention to have end portions of unequal length. In any of these configurations, the intermediate portion has a length sufficient to function as a replacement tendon or ligament and the end portion or end portions have a length sufficient to facilitate passing the graft into a bone tunnel. The intermediate portion preferably has a length in the range of 20-80% of the overall graft length, more preferably 25-75%, more preferably 40-60%.

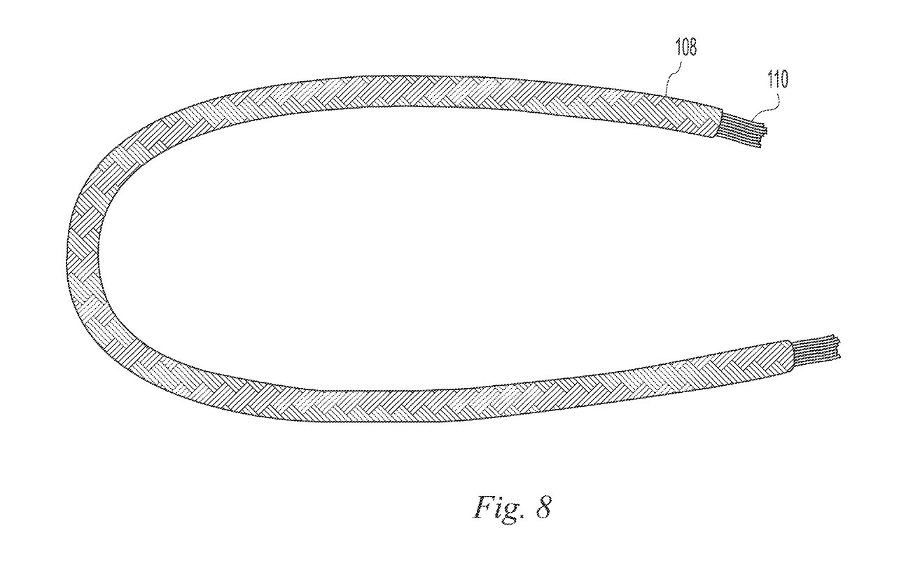

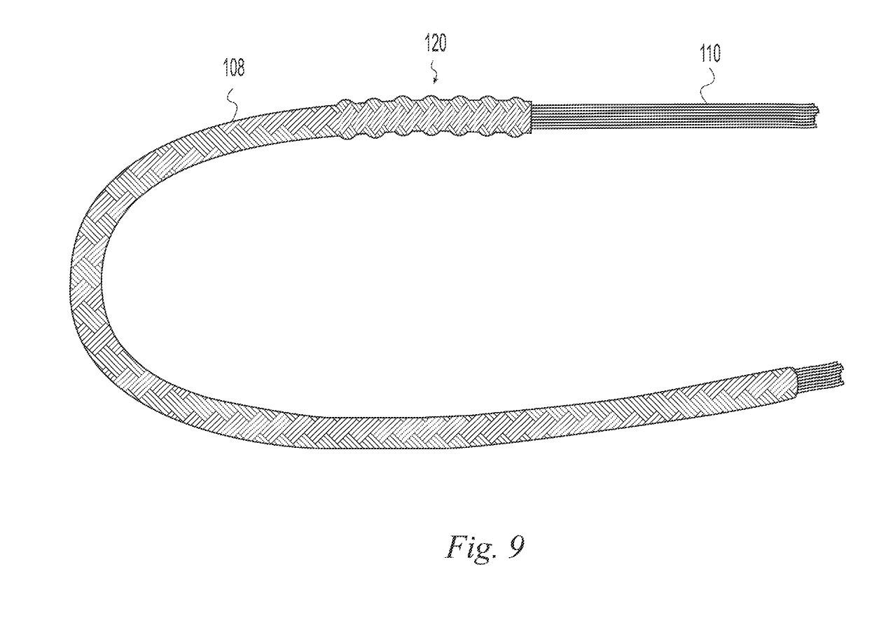

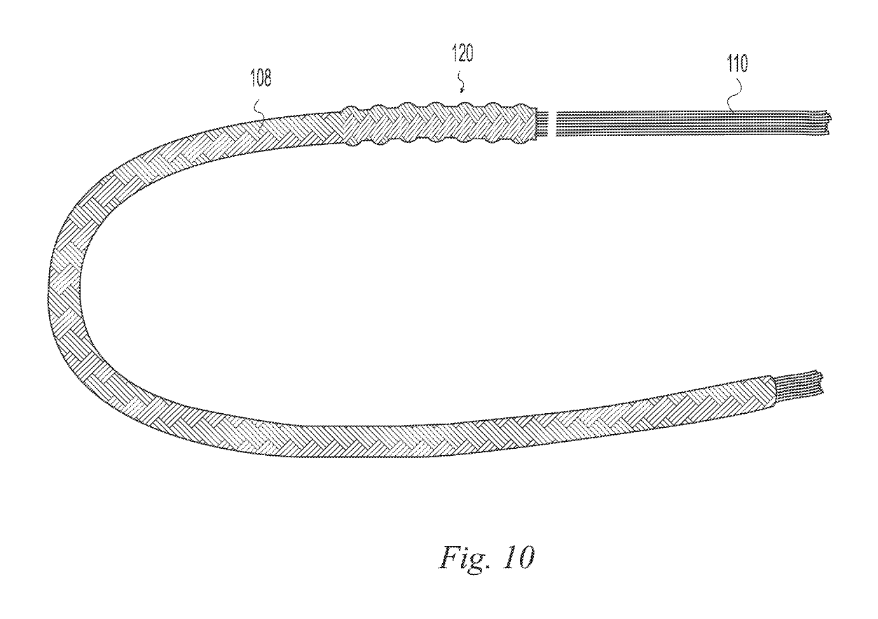

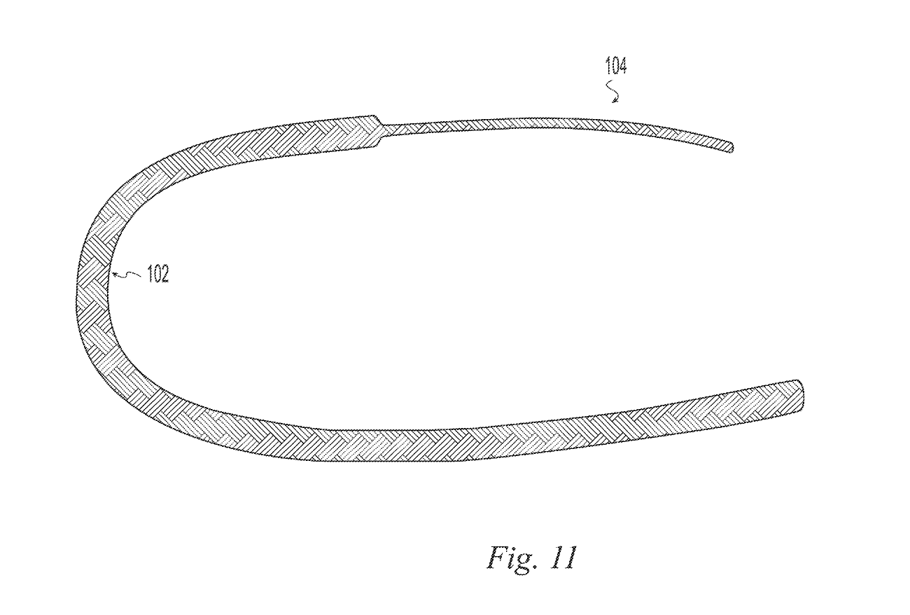

FIGS. 8-11 illustrate an exemplary method of forming the soft tissue graft 100 of FIGS. 5-7. Referring to FIG. 8, a precursor to the graft is provided with a braided jacket 108 and a fibrous core 110. For example, the jacket 108 may be braided over the core 110 as is known in the art. The precursor may be produced in lengths sufficient to form several grafts and then cut into a portion as shown in FIG. 8 for further conversion. Referring to FIG. 9, the jacket 108 has been positioned back from one end into a longitudinally compressed portion 120 exposing a portion of core 110. Referring to FIG. 10, the core 110 has been severed and removed at one end for a length corresponding to a desired length of an end portion 104. Referring to FIG. 11, the jacket has been returned to its original position and tensioned so that it forms an end portion 104 having a smaller cross-sectional area than the intermediate portion 102. If it is desired to have an end portion 106 at the other end of the graft, the process is repeated for the other end.

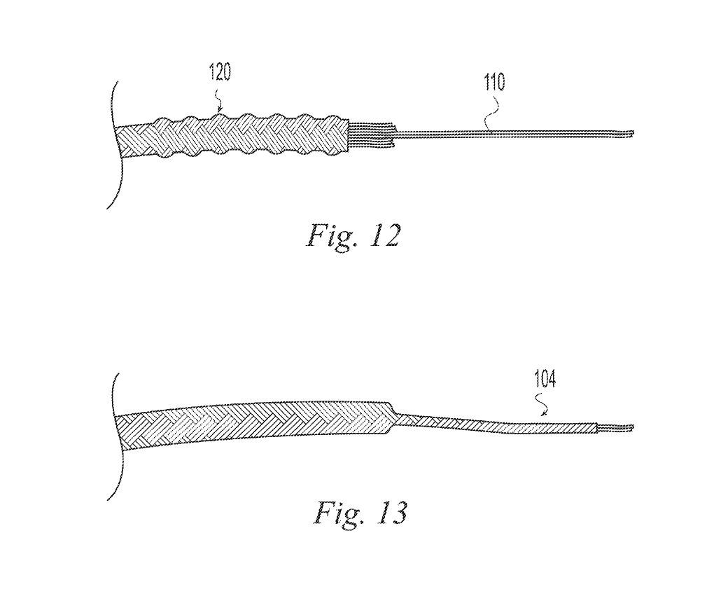

FIGS. 12 and 13 illustrate an alternative construction for the end portion in which some, but not all, of the core is removed from the end portion. This construction may be utilized for various advantages including increased strength in the end portion, allowing tensioning of the core, and providing a way to control the relative position of the core and jacket. Some of these advantages may be enhanced in constructions in which the core is a unitary structure or in which the core is formed of a woven, braided, or otherwise interconnected network.

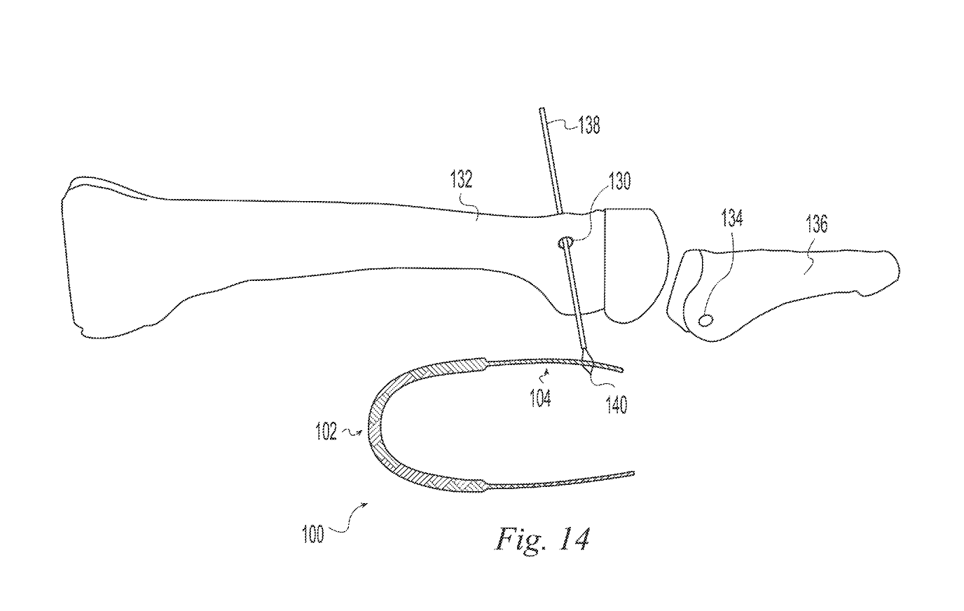

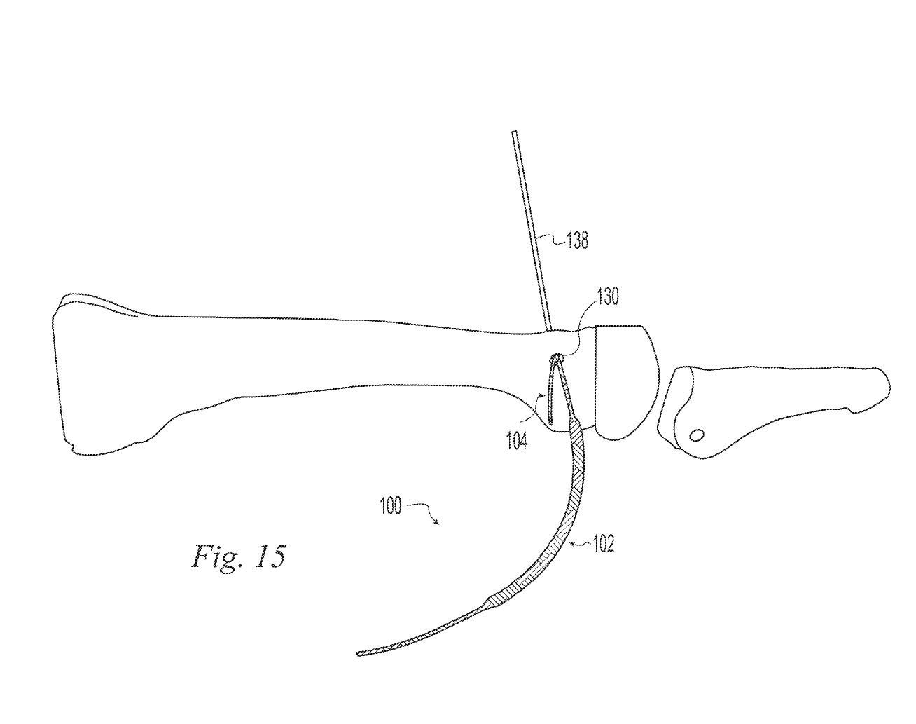

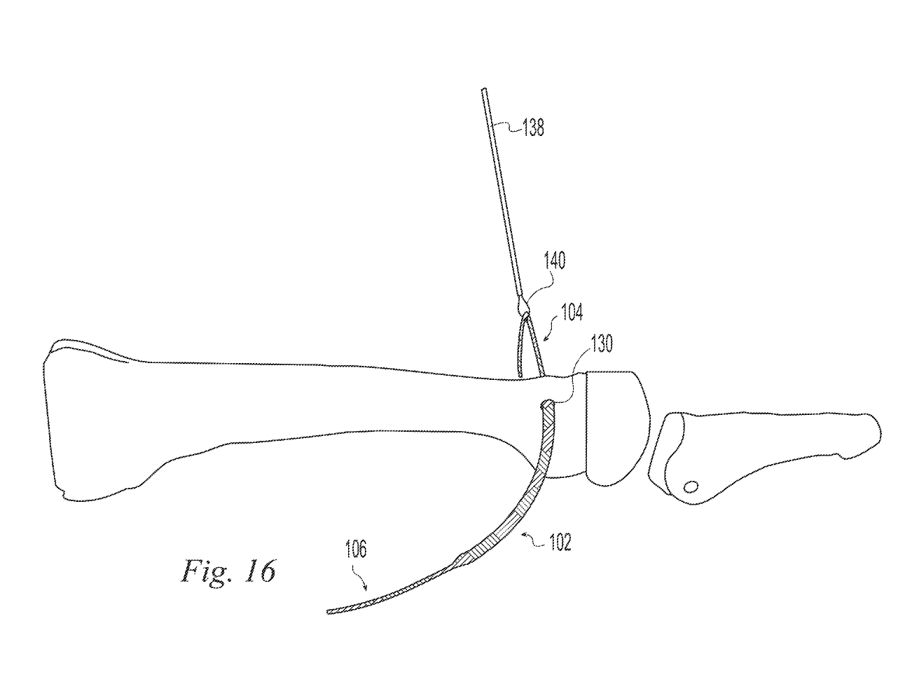

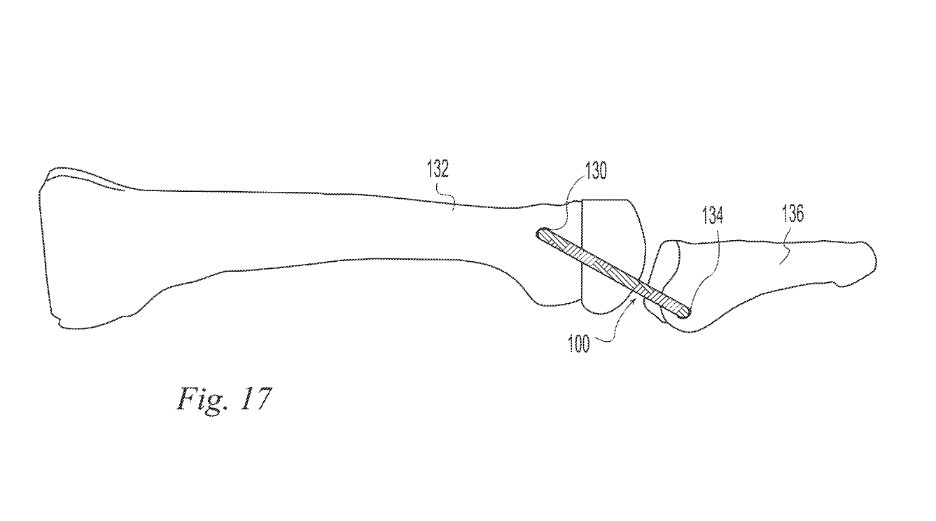

FIGS. 14-17 illustrate an illustrative surgical method utilizing the soft tissue graft of FIGS. 5-7. In the illustrative example of FIGS. 14-17, the graft is shown in use to reconstruct a collateral ligament of a joint of an extremity of a human patient. In this example, the ligament is the lateral PCL of an MTP joint of a human foot. A tunnel 130 has been formed in the metatarsal bone 132 approximately at the anatomic origin of the lateral PCL. A tunnel 134 has been formed in the proximal phalanx 136 approximately at the insertion of the lateral PCL. A graft passer 138 having a loop 140 is passed through the metatarsal tunnel 130 and an end portion 104 of the soft tissue graft 100 is inserted through the loop 140. Referring to FIG. 15, the graft passer 138 has been withdrawn pulling the end portion 104 into the tunnel 130. The tunnel 130 is sized for a press fit with the intermediate portion 102 of the soft tissue graft 100. Because the end portion 104 has a reduced cross sectional area, it is able to fold around the loop 140 and be pulled through the tunnel 130 in a doubled over configuration which allows it to be pulled through the tunnel 130 and without becoming disengaged from the loop 140. Referring to FIG. 16, the end portion 104 has been pulled through the bone and may be advanced further with the graft passer or by gripping the end portion directly or with another instrument to position the end of the intermediate portion in the bone tunnel. Referring to FIG. 17, the other end of the soft tissue graft 100 has been passed through the tunnel 134 in the proximal phalanx 136 and both ends of the soft tissue graft 100 have been secured to complete the lateral PCL reconstruction. Excess length of the soft tissue graft 100, including portions of the ends 104, 106 have been cut and removed from the surgical site.

The illustrative examples have depicted a soft tissue graft constructed and used for soft tissue reconstruction of an MTP joint of a human foot. The graft and methods of the present invention are suitable for grafting at other locations within a patient's body including, but not limited to, the MCP joints of the human hand.

What is claimed is:

1. A soft tissue graft comprising an elongated flexible body comprising:
a continuous, elongated, flexible, tubular jacket extending from a first end to a second end and having a uniform construction throughout its length, the jacket defining a first end portion adjacent the first end and an intermediate portion extending from the first end portion, the intermediate portion having a length in the range of 20-80% of the overall graft length, the first end portion having a first end portion cross-sectional area and the intermediate portion having an intermediate portion cross-sectional area, the cross-sectional area of the intermediate portion being greater than the cross-sectional area of first end portion; and
an elongated, flexible core positioned within the tubular jacket in the intermediate portion.

2. The soft tissue graft of claim 1 further comprising a second end portion adjacent the second end, the second end portion having a second end portion cross-sectional area less than the intermediate portion cross-sectional area, the intermediate portion extending between the first and second end portions.

3. The soft tissue graft of claim 1 wherein the jacket and core are separate, unjoined members.

4. The soft tissue graft of claim 1 wherein the first end portion is more flexible than the intermediate portion.

5. The soft tissue graft of claim 4 wherein the first end portion may be doubled over on itself and the doubled over cross-sectional area of first end portion is less than or equal to the cross-sectional area of the intermediate portion.

6. The soft tissue graft of claim 2 wherein a first portion of the core is in the intermediate portion of the jacket, a second portion of the core extends into the first end portion, and a third portion of the core extends into the second end portion, each of the core portions having a cross-sectional area, the first and second portions of the core each having a cross-sectional area smaller than the cross-sectional area of the first portion of the core.

7. The soft tissue graft of claim 2 wherein the core is within the intermediate portion of the jacket and absent in the end portions of the jacket.

8. The soft tissue graft of claim 1 wherein the jacket comprises braided biocompatible fibers.

9. The soft tissue graft of claim 1 wherein the core comprises unbraided biocompatible fibers.

10. A method of forming a soft tissue graft, the method comprising:
providing a graft precursor having a continuous, elongated, flexible, tubular jacket extending from a first end to a second end and having a uniform construction throughout its length surrounding a flexible core;
exposing the core at a first end portion;
removing at least a portion of the core from the first end portion;
tensioning the jacket to form a reduced cross section in the first end portion where the core has been removed.

11. The method of claim 10 wherein the jacket is a textile comprising a network of biocompatible fibers.

12. The method of claim 11 wherein the core comprises parallel biocompatible fibers.

13. The method of claim 10 wherein exposing the core at a first end portion comprises compressing the jacket away from the end portion.

14. The method of claim 10 wherein removing a portion of the core from the first end portion comprises removing the core completely from the first end portion.

15. The method of claim 10 further comprising:
exposing the core at a second end portion;
removing at least a portion of the core from the second end portion;
tensioning the jacket to form a reduced cross section in the second end portion where the core has been removed.

16. A method of reconstructing a connective soft tissue adjacent a joint, the method comprising:
folding a first end portion of a graft over a graft passer, the first end portion having a cross-sectional area less than an intermediate portion of the graft;
passing the folded first end portion into a first bone tunnel; and
advancing the folded first end portion to position a portion of the intermediate portion in the first bone tunnel.

17. The method of claim 16 further comprising:
folding a second end portion of a graft over a graft passer, the second end portion having a cross-sectional area less than an intermediate portion of the graft;
passing the folded second end portion into a second bone tunnel; and
advancing the folded second end portion to position a portion of the intermediate portion in the second bone tunnel.

18. The method of claim 17 wherein the first bone tunnel is formed in a metatarsal bone of a human foot and the second bone tunnel is formed in a proximal phalanx of a human foot.

19. The method of claim 16 further comprising:
securing the graft in the bone tunnel.

20. The method of claim 16 wherein the first bone tunnel is sized to fit closely around the intermediate portion.
